(19) United States
(12) Patent Application Publication
(10) Pub. No.: US 2012/0259196 A1
(43) Pub. Date: Oct. 11, 2012

(54) MEDICAL IMAGE DIAGNOSTIC APPARATUS AND CONTROL METHOD

(75) Inventors: Takashi Tanaka, Nasushiobara-shi (JP); Katsuhito Morino, Utsunomiya-shi (JP); Hiroyuki Onuki, Nasushiobara-shi (JP); Tatsuya Watanabe, Nasushiobara-shi (JP); Shinya Kawanabe, Nasushiobara-shi (JP); Risa Onishi, Nasushiobara-shi (JP); Masao Yamahana, Nasushiobara-shi (JP)

(73) Assignees: Toshiba Medical Systems Corporation, Otawara-shi (JP); Kabushiki Kaisha Toshiba, Tokyo (JP)

(21) Appl. No.: 13/438,102

(22) Filed: Apr. 3, 2012

(30) Foreign Application Priority Data
Apr. 6, 2011 (JP) 2011-084330
Feb. 28, 2012 (JP) 2012-041290

(51) Int. Cl. A61B 6/00; A61B 5/055

(52) U.S. Cl. 600/407; 600/411

(57) ABSTRACT

A medical image diagnostic apparatus according to an embodiment includes a simplified positron emission computed tomography (PET) image data generating unit, a PET image data generating unit, and a display. The simplified PET image data generating unit generates simplified PET image data based on information obtained by projecting the position of a generation source of a gamma ray emitted from a subject to whom a radioisotope is administered onto a predetermined projection surface in a predetermined direction. The PET image data generating unit generates, based on an evaluation result of the simplified PET image data, PET image data by using projection data in the PET imaging mode generated based on a detection result of the gamma ray emitted from the subject. The display displays the simplified PET image data and the PET image data.

### CROSS-REFERENCE TO RELATED APPLICATIONS

This application is based upon and claims the benefit of priority from Japanese Patent Application No. 2011-084330, filed on Apr. 6, 2011; and Japanese Patent Application No. 2012-041290, filed on Feb. 28, 2012, the entire contents of all of which are incorporated herein by reference.

### FIELD

Embodiments described herein relate generally to a medical image diagnostic apparatus and a control method.

### BACKGROUND

Medical imaging diagnosis using an X-ray diagnostic apparatus, a magnetic resonance imaging (MRI) apparatus, an X-ray computed tomography (CT) apparatus, a nuclear medicine imaging apparatus, and the like has progressed rapidly in association with development in computer technology. Medical imaging diagnosis is necessary for medical care these days.

Such X-ray diagnostic apparatuses and X-ray CT apparatuses described above are used for so-called form diagnosis in which diagnosis is performed by visualizing an outline of an organ, a tumor, and the like. By contrast, such nuclear medicine imaging apparatuses described above enable function diagnosis for a subject by measuring a gamma ray emitted from a radioisotope selectively introduced into a body tissue or from a labeled compound labeled with a radioisotope outside of the body, and imaging dose distribution of the gamma ray thus measured.

Examples of such nuclear medicine imaging apparatuses in clinical use include a gamma camera, a single photon emission computed tomography (SPECT) apparatus, and a positron emission computed tomography (PET) apparatus.

A gamma camera measures a gamma ray emitted from inside of the subject to whom a medical agent labeled with a radioisotope (hereinafter, referred to as a radioisotope) is administered by using a flat panel detector arranged facing the subject. The gamma camera is an apparatus that generates the distribution of the radioisotope projected onto the flat panel detector as two-dimensional image data (gamma image data). The gamma camera specifies the incident direction of the gamma ray by using a collimator attached to the front of the flat panel detector.

An SPECT apparatus causes a flat panel detector similar to that of the gamma camera to move around the subject to whom the radioisotope is administered. Alternatively, the SPECT apparatus arranges a plurality of flat panel detectors similar to that of the gamma camera around the subject to whom the radioisotope is administered. With this configuration, the SPECT apparatus performs reconstruction processing similar to that in the X-ray CT apparatus on gamma-ray information detected in a plurality of directions with respect to the subject, thereby generating image data (SPECT image data).

By contrast, a PET apparatus detects a pair of gamma rays emitted when a positron binds to an electron to annihilate from the subject to whom a radioisotope labeled with a positron-emitting radionuclide is administered by using a ring detector arranged around the subject. The PET apparatus reconstructs a pair of pieces of gamma ray information detected by the detector, thereby generating image data (PET image data).

In recent years, so-called X-ray CT-combined positron CT apparatuses (hereinafter, referred to as PET-CT apparatuses) obtained by combining an X-ray CT apparatus and a PET apparatus have been developed. With such a PET-CT apparatus, it is possible to perform form diagnosis and function diagnosis on a single subject efficiently. Furthermore, with the PET-CT apparatus, when generating PET image data by reconstructing projection data acquired by PET imaging, it is possible to obtain excellent PET image data by correcting the projection data using attenuation correction data (attenuation map) generated based on the pixel value of X-ray CT image data.

To acquire the PET image data, projection data such as a sinogram is generated based on information on the detection direction and the detection position of the gamma ray emitted from the radioisotope administered to the subject. Subsequently, the projection data is reconstructed, whereby PET image data is generated. By displaying the PET image data thus obtained on a display and the like, it is determined whether or how much influence (hereinafter, referred to as an image quality deterioration factor) that deteriorates the image quality of the PET image data, such as body movement, affects the projection data. If an unacceptable image quality deterioration factor is present, the projection data is reacquired.

In such a conventional method, however, it takes long time to reconstruct the projection data. Therefore, to reacquire the projection data, it is necessary to administer the radioisotope to the subject again. Furthermore, when the image quality deterioration factor is confirmed to be present based on the observation of the PET image data, it is often the case that the subject has already left a laboratory. In this case, it is necessary to ask the subject to come back to the hospital. Such readministration of the radioisotope and a request for coming back to the hospital to the subject not only decrease the examination efficiency significantly, but also increase the burden on the subject.

### BRIEF DESCRIPTION OF THE DRAWINGS

FIG. 1 is a block diagram of an entire configuration of a medical image diagnostic apparatus according to an embodiment;

FIG. 2 is a block diagram of a specific configuration of an X-ray CT imaging unit included in the medical image diagnostic apparatus in the present embodiment;

FIG. 3 is a block diagram of a specific configuration of a PET imaging unit included in the medical image diagnostic apparatus in the present embodiment;

FIG. 4 is a schematic of a specific structure of a detector module included in the PET imaging unit in the present embodiment;

FIG. 5A and FIG. 5B are schematics illustrating the detection directions and the detection positions of gamma rays in a simplified PET imaging mode in the present embodiment;

FIG. 6 is a schematic for explaining evaluation image data generated by an image data synthesis unit in the present embodiment;

FIG. 7 is a schematic for explaining an X-ray CT gantry and a PET gantry moved by a movement mechanical unit in the present embodiment;

FIG. 8 is a flowchart of a generation process of PET image data in the present embodiment;

FIG. 9 is a block diagram of an entire configuration of a medical image diagnostic apparatus according to a modification of the present embodiment;

FIG. 10A and FIG. 10B are schematics for explaining a specific example of an image quality deterioration factor determined by evaluation image data in the present embodiment and the modification thereof; and

FIG. 11 and FIG. 12 are schematics for explaining a modification of simplified PET image data generation processing.

### DETAILED DESCRIPTION

A medical image diagnostic apparatus according to an embodiment includes a simplified positron emission computed tomography (PET) image data generating unit, a PET image data generating unit, and a display. The simplified PET image data generating unit generates simplified PET image data based on information obtained by projecting the position of a generation source of a gamma ray emitted from a subject to whom a radioisotope is administered onto a predetermined projection surface in a predetermined direction. The PET image data generating unit generates, based on an evaluation result of the simplified PET image data, PET image data by using projection data in the PET imaging mode generated based on a detection result of the gamma ray emitted from the subject. The display displays the simplified PET image data and the PET image data.

Exemplary embodiments of a medical image diagnostic apparatus are described below with reference to the accompanying drawings.

The medical image diagnostic apparatus according to the present embodiment described below generates a scanogram based on projection data in a scanogram imaging mode acquired by X-ray imaging of a subject with an X-ray tube and an X-ray detector fixed at predetermined positions. Subsequently, the medical image diagnostic apparatus generates projection data in a positron emission computed tomography (PET) imaging mode based on the detection direction and the detection position of a gamma ray emitted from inside of the body of the subject to whom a radioisotope is administered. At this time, the medical image diagnostic apparatus generates not only the projection data in the PET imaging mode, but also simplified PET image data based on the detection position of a gamma ray detected in a direction nearly equal to an X-ray irradiation direction (specifically, direction of the center of the X-ray irradiation direction) in the X-ray imaging extracted from the detection result of the gamma ray. The medical image diagnostic apparatus then superimposes the simplified PET image data thus obtained on the scanogram described above, thereby generating evaluation image data. Subsequently, by observing the evaluation image data in which an observation point corresponding to the detection position of the gamma ray is displayed in real time on a display, if it is confirmed that no influence serving as an image quality deterioration factor, such as body movement and leakage of a medical agent, is included in the projection data in the PET imaging mode, the medical image diagnostic apparatus reconstructs the projection data (projection data in the PET imaging mode), thereby generating diagnostic PET image data.

In the embodiment below, an explanation will be made of the medical image diagnostic apparatus capable of generating a scanogram serving as form image data based on the projection data in the scanogram imaging mode and generating PET image data serving as function image data based on the projection data in the PET imaging mode. Alternatively, in the embodiment below, three-dimensional image data generated based on projection data in an X-ray computed tomography (CT) imaging mode obtained by rotating the X-ray tube and the X-ray detector around the subject at high speed or multi-planer reconstruction (MPR) image data at a predetermined section (e.g., a coronal section) may be used as the form image data, for example.

Configuration of the Apparatus

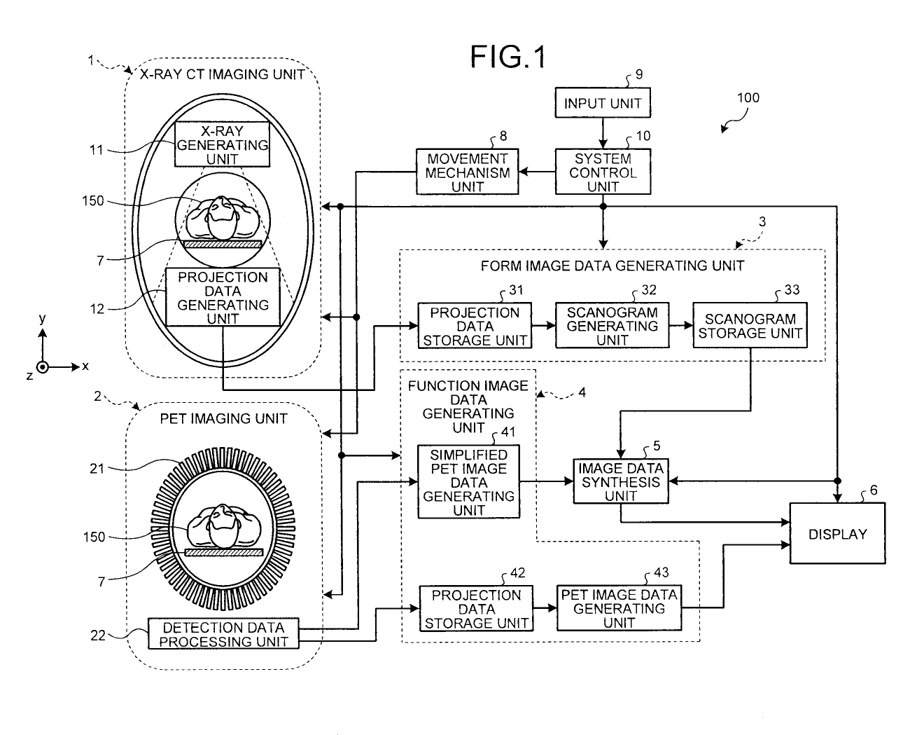

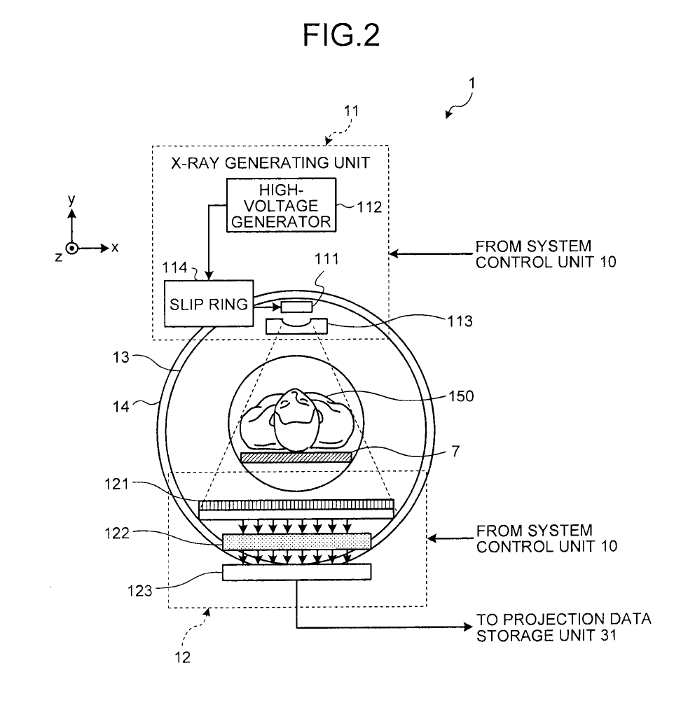

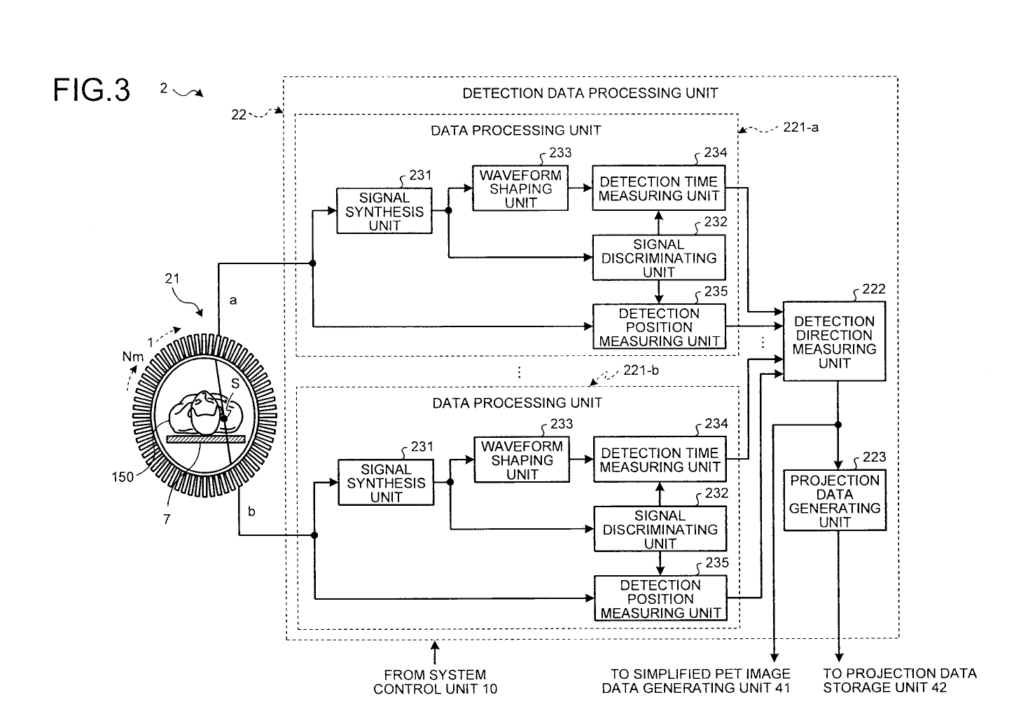

A configuration of the medical image diagnostic apparatus according to the embodiment of the present description will now be described with reference to FIG. 1 to FIG. 7. FIG. 1 is a block diagram of an entire configuration of the medical image diagnostic apparatus according to the present embodiment. FIG. 2 is a block diagram of a specific configuration of an X-ray CT imaging unit included in the medical image diagnostic apparatus according to the present embodiment. FIG. 3 is a block diagram of a specific configuration of a PET imaging unit included in the medical image diagnostic apparatus according to the present embodiment.

A medical image diagnostic apparatus 100 according to the present embodiment illustrated in FIG. 1 includes an X-ray CT imaging unit 1, a PET imaging unit 2, a form image data generating unit 3, a function image data generating unit 4, an image data synthesis unit 5, and a display 6. The X-ray CT imaging unit 1 acquires projection data in the X-ray CT imaging mode from a subject 150. The X-ray CT imaging unit 1 according to the present embodiment causes a projection data generating unit 12 to detect an X-ray that is output from an X-ray generating unit 11 of a rotating gantry fixed at a predetermined position and that passes through the subject 150, thereby generating projection data in the scanogram imaging mode. The PET imaging unit 2 causes detector modules 21 arranged around the subject 150 to detect a pair of gamma rays emitted from inside of the body of the subject 150 to whom the radioisotope is administered, thereby generating projection data in the PET imaging mode based on the detection directions and the detection positions of the pair of gamma rays. The form image data generating unit 3 uses the projection data in the scanogram imaging mode generated by the X-ray CT imaging unit 1 to generate a scanogram serving as form image data. The function image data generating unit 4 generates simplified PET image data based on the detection position of a gamma ray detected in a direction nearly equal to an X-ray irradiation direction (specifically, direction of the center of the X-ray irradiation direction) in the X-ray CT imaging unit 1. Furthermore, the function image data generating unit 4 generates PET image data serving as function image data based on the projection data in the PET imaging mode generated by the PET imaging unit 2. The image data synthesis unit 5 superimposes the simplified PET image data on the scanogram, thereby generating evaluation image data used for evaluating an image quality deterioration factor included in the projection data in the PET imaging mode. The display 6 displays the evaluation image data generated by the image data synthesis unit 5 and the PET image data generated by the function image data generating unit 4.

The medical image diagnostic apparatus 100 further includes a couchtop 7, a movement mechanical unit 8, an input unit 9, and a system control unit 10. The couchtop 7 on which the subject 150 is placed is fixed to a couch, which is not illustrated. The movement mechanical unit 8 moves an X-ray CT gantry including the X-ray CT imaging unit 1 and a PET gantry including the PET imaging unit 2 (neither of which is illustrated) in the body axis direction (z-direction in FIG. 1), thereby putting a region to be examined of the subject 150 in each imaging field. The input unit 9 receives subject information, selection of the scanogram imaging mode, the simplified PET imaging mode, and the PET imaging mode, setting of imaging conditions in these imaging modes, setting of generating conditions and display conditions for the scanogram, the simplified PET image data, and the PET image data, and various types of command signals, for example. The system control unit 10 collectively controls the units included in the medical image diagnostic apparatus 100.

The configurations and the functions of the units included in the medical image diagnostic apparatus 100 will now be described in greater detail.

As illustrated in FIG. 2, the X-ray CT imaging unit 1 illustrated in FIG. 1 includes the X-ray generating unit 11, the projection data generating unit 12, a rotating gantry 13, and a fixed gantry 14. The X-ray generating unit 11 includes an X-ray tube 111, a high-voltage generator 112, an X-ray beam limiter 113, and a slip ring 114. The X-ray tube 111 irradiates the subject 150 with an X-ray. The high-voltage generator 112 generates high voltage to be applied between an anode and a cathode of the X-ray tube 111. The X-ray beam limiter 113 specifies an area irradiated with the X-ray output from the X-ray tube 111. The slip ring 114 supplies predetermined electric power to the rotating gantry 13.

The X-ray tube 111 is a vacuum tube that generates an X-ray. The X-ray tube 111 causes an electron accelerated by high voltage supplied from the high-voltage generator 112 to collide with a tungsten target, thereby outputting an X-ray. The X-ray beam limiter 113 is arranged between the X-ray tube 111 and the subject 150. The X-ray beam limiter 113 has a function to limit the X-ray output from the X-ray tube 111 to a predetermined imaging area and a function to set irradiation intensity distribution of the X-ray for the subject 150. The X-ray beam limiter 113, for example, forms an X-ray beam output from the X-ray tube 111 into a cone beam shape or a fan beam shape corresponding to the imaging area.

The projection data generating unit 12 includes an X-ray detector 121, a data acquiring unit 122, and a data transmission circuit 123. The X-ray detector 121 detects the X-ray passing through the subject 150. The data acquiring unit 122 is a data acquisition system (DAS) unit, and performs current/voltage conversion and analog/digital (A/D) conversion on detection signals of a plurality of channels output from the X-ray detector 121. Hereinafter, the data acquiring unit 122 is referred to as a DAS unit 122. The data transmission circuit 123 performs parallel/serial conversion, electrical/optical/electrical conversion, and serial/parallel conversion on an output signal from the DAS unit 122.

The X-ray detector 121 of the projection data generating unit 12 includes a plurality of X-ray detecting elements in a two-dimensional array, which are not illustrated, for example. Each of the X-ray detecting elements is composed of a scintillator that converts an X-ray into light and a photo diode that converts light into an electrical signal. The X-ray detecting elements are attached to the rotating gantry 13 along a circular arc about a focal point of the X-ray tube 111.

The DAS unit 122 performs current/voltage conversion and A/D conversion on the detection signal output from the X-ray detector 121. The data transmission circuit 123 includes a parallel/serial converter, an electrical/optical/electrical converter, and a serial/parallel converter, which are not illustrated. The detection signal output from the DAS unit 122 is converted into time-series projection data of one channel by the parallel/serial converter attached to the rotating gantry 13. The projection data is then supplied to the serial/parallel converter attached to the fixed gantry 14 through optical communications using the electrical/optical/electrical converter.

Subsequently, the projection data of one channel is converted into projection data of a plurality of channels by the serial/parallel converter. The projection data is then stored in a projection data storage unit 31 of the form image data generating unit 3 as projection data in the scanogram imaging mode.

The data transmission method described above can be replaced by another method as long as it is possible to transmit a signal between the projection data generating unit 12 provided to the rotating gantry 13 and the form image data generating unit 3 provided outside of the fixed gantry 14. A device, such as the slip ring described above, may be used, for example.

In this case, the X-ray tube 111 and the X-ray beam limiter 113 of the X-ray generating unit 11 and the X-ray detector 121 and the DAS unit 122 of the projection data generating unit 12 are attached to the rotating gantry 13 in a manner facing each other with the subject 150 interposed therebetween. As illustrated in FIG. 2, in the scanogram imaging mode, the rotating gantry 13 is fixed to a predetermined position such that the X-ray tube 111 and the X-ray beam limiter 113 are arranged above the subject 150 and that the X-ray detector 121 is arranged below the subject 150.

As illustrated in FIG. 3, the PET imaging unit 2 in FIG. 1 includes the detector modules 21 and a detection data processing unit 22. The detector modules 21 are arranged concentrically around the subject 150, and detect a pair of gamma rays emitted from inside of the body of the subject 150 to whom the radioisotope is administered. The detection data processing unit 22 discriminates the gamma ray thus detected from noise, measures the detection time and the detection position of the gamma ray, and measures the detection direction based on the detection positions of a pair of gamma rays measured simultaneously. Furthermore, the detection data processing unit 22 cumulatively calculates the count value of the gamma ray in a predetermined time period in a manner corresponding to the gamma-ray detection position and the gamma-ray detection direction, thereby generating projection data in the PET imaging mode. A line segment connecting the detection positions of the pair of gamma rays measured simultaneously is referred to as a line of response (LOR). The generation source of the pair of gamma rays emitted from inside of the body of the subject 150 is positioned on the LOR.

The detector modules 21 (21-1 to 21-Nm) are arranged concentrically around the subject 150 positioned in the imaging field of the PET imaging unit 2 in a state being placed on the couchtop 7. The gamma ray emitted from the subject 150 is converted into visible light temporarily, and is converted into an electrical signal (detection signal) by the detector modules 21.

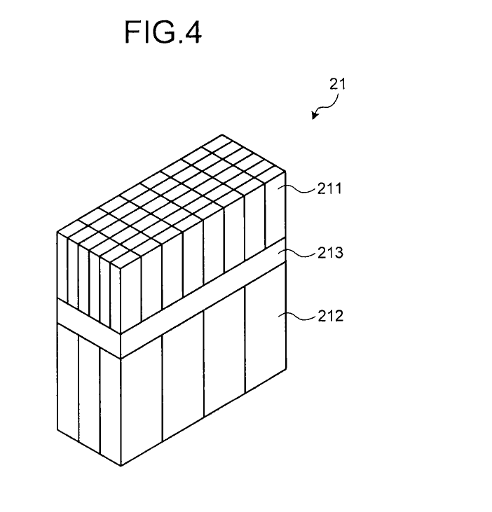

FIG. 4 is a schematic of a specific structure of the detector module included in the PET imaging unit according to the present embodiment. As illustrated in FIG. 4, each of the detector modules 21-1 to 21-Nm includes strip-shaped scintillators 211, photomultipliers 212, and a light guide 213. The scintillator 211 detects the gamma ray emitted from the subject 150, and converts the gamma ray into visible light. The photomultiplier 212 converts the visible light converted by the scintillator 211 into an electrical signal, and amplifies the weak electrical signal thus converted. The light guide 213 transmits the visible light output from the scintillator 211 to the photomultiplier 212.

The scintillator 211 is made of bismuth germanid (BGO: (Bi₄Ge₃O₁₂)), thallium-activated sodium iodide (NaI(Tl)), barium fluoride (BaF₂), or the like. In particular, BGO, which has high gamma-ray photoelectric absorption rate per unit volume, and BaF₂, which has excellent responsibility, are suitably used for the detector modules 21 of the PET imaging unit 2.

The photomultiplier 212 amplifies hundreds of photons into 10⁷ to 10¹⁰ electrons, for example, and acquires the electrons to an anode serving as an output stage, thereby converting the electrons into an electrical signal. The photomultiplier 212 includes a photocathode and an electron multiplier, which are not illustrated. The photocathode is made of a multi-alkali material whose wavelength characteristics are nearly equal to those of the emission wavelength of the scintillator 211 or of a bi-alkali material activated by oxygen and cesium. The rate of the number of generated photoelectrons to the number of incident photons is usually 20% to 30%. The electron multiplier is formed of a multistage electrode arranged along a transmission path of the electrons and an anode to which the electrons thus amplified are acquired based on a secondary electron emission phenomenon. The amplification factor per one stage in the case of tube voltage of 200 V to 300 V is approximately 5 times. Therefore, to obtain amplification factor of 10⁷ described above, an electrode of approximately 10 stages is provided.

The light guide 213 optically couples the scintillators 211 and the photomultiplier 212. The light guide 213 is made of a plastic material, which has excellent optical transparency, so as to transmit the visible light output from the scintillator 211 to the photomultiplier 212 efficiently.

Referring back to FIG. 3, the detection data processing unit 22 of the PET imaging unit 2 includes data processing units 221-1 to 221-Nm of Nm channels connected to the detector modules 21-1 to 21-Nm, respectively. Furthermore, the detection data processing unit 22 includes a detection direction measuring unit 222 that measures the detection direction of the gamma ray based on detection position information of the gamma ray output from the data processing units 221-1 to 221-Nm. Moreover, the detection data processing unit 22 includes a projection data generating unit 223 that cumulatively adds the count value of the detection signal in a predetermined time period sequentially in a manner corresponding to the gamma-ray detection position and the gamma-ray detection direction, thereby generating projection data in the PET imaging mode.

In the present embodiment, it is assumed that a pair of gamma rays emitted from a radioisotope S administered to the subject 150 is detected by detector modules 21-a and 21-b. Therefore, only data processing units 221-a and 221-b that are connected to the detector modules 21-a and 21-b, respectively, are illustrated.

Each of the data processing units 221-a and 221-b of the detection data processing unit 22 includes a signal synthesis unit 231, a signal discriminating unit 232, a waveform shaping unit 233, a detection time measuring unit 234, and a detection position measuring unit 235. The signal synthesis unit 231 additively synthesizes the detection signals of a plurality of channels supplied from the photomultipliers 212 of the detector module 21-a or 21-b. The signal discriminating unit 232 uses the detection signal synthesized by the signal synthesis unit 231 to discriminate the detection signal attributable to the gamma ray from noise based on each peak value. The waveform shaping unit 233 shapes the detection signal synthesized and output from the signal synthesis unit 231 into a square wave. The detection time measuring unit 234 measures the detection time of the gamma ray corresponding of the detection signal discriminated by the signal discriminating unit 232 based on a front edge of the square wave supplied from the waveform shaping unit 233, for example. The detection position measuring unit 235 measures the detection position of the gamma ray corresponding of the detection signal discriminated by the signal discriminating unit 232 based on the detection signals of the channels supplied from the photomultipliers 212 of the detector module 21-a or 21-b. For example, the detection position measuring unit 235 calculates the center of gravity using Anger logic, thereby specifying the scintillator 211 that emits a plurality of photons attributable to one gamma ray. The detection position measuring unit 235 then determines the position of the scintillator 211 thus specified to be the detection position of the gamma ray. Note that the specific configurations and functions of the units constituting the data processing unit 221 are disclosed in Japanese Patent Application Laid-open No. 2007-107995, for example. Therefore, detailed explanations thereof will be omitted.

Based on information on the detection time of the gamma ray and the detection position of the gamma ray supplied from the detection time measuring unit 234 and the detection position measuring unit 235, respectively, included in each of the data processing units 221-1 to 221-Nm, the detection direction measuring unit 222 of the detection data processing unit 22 measures the detection direction of the gamma ray emitted from inside of the body of the subject 150. For example, the detection direction measuring unit 222 determines two detection positions the difference between the detection times of which is a predetermined duration to be positions at which a pair of gamma rays emitted from inside of the body of the subject 150 is detected nearly simultaneously. Subsequently, the detection direction measuring unit 222 determines the line segment connecting the two detection positions to be an LOR, for example, and measures the direction of the LOR as the detection direction of the gamma rays.

The projection data generating unit 223 generates projection data in the PET imaging mode based on the detection result of the gamma ray emitted from the subject 150. The projection data generating unit 223 includes a storage circuit (not illustrated) having a cumulative calculation function. The projection data generating unit 223 stores the count value of the detection signal supplied from the detection direction measuring unit 222 in the storage circuit in a manner corresponding to the detection position and the detection direction of the gamma ray. Every time the detector module 21-a and the detector module 21-b detect gamma rays in a predetermined time period, for example, the count value of the detection signal is cumulatively added in an address of the storage circuit corresponding to the detection position and the detection direction.

Furthermore, even if other detection modules 21 different from the detector module 21-a and the detector module 21-b detect a pair of gamma rays, the detection data processing unit 22 measures the detection position and the detection direction of the gamma rays similarly to the method described above. The detection data processing unit 22 then cumulatively adds the count value of the detection signal in an address of the storage circuit corresponding to the detection position and the detection direction of the gamma ray. In other words, the count value of the gamma ray sequentially detected in the predetermined time period is cumulatively added in the address of the storage circuit corresponding to the detection position and the detection direction. As a result, projection data in the PET imaging mode is generated.

The form image data generating unit 3 illustrated in FIG. 1 includes a projection data storage unit 31, a scanogram generating unit 32, and a scanogram storage unit 33. Projection data in the scanogram imaging mode is acquired by X-ray imaging performed while sliding the X-ray CT gantry, which will be described later, in the body axis direction (z-direction) of the subject 150 with the rotating gantry 13 fixed to a predetermined position. The projection data in the scanogram imaging mode is provided with identification information of the imaging mode, imaging position information (that is, positional information of the X-ray CT gantry), and the like as additional information, and is stored in the projection data storage unit 31.

Based on the imaging position information serving as the additional information, the scanogram generating unit 32 synthesizes the projection data in the scanogram imaging mode read from the projection data storage unit 31 based on the identification information of the imaging mode. Thus, the scanogram generating unit 32 generates a scanogram having a broad area in the body axis direction. The scanogram thus obtained is stored in the scanogram storage unit 33 temporarily. The scanogram generated at this time is similar to transparent image data acquired by a typical X-ray diagnostic apparatus.

The function image data generating unit 4 includes a simplified PET image data generating unit 41, a projection data storage unit 42, and a PET image data generating unit 43.

The simplified PET image data generating unit 41 generates simplified PET image data based on information obtained by projecting the position of the generation source of the gamma ray emitted from the subject 150 to whom the radioisotope is administered onto a predetermined projection surface in a predetermined direction. In the present embodiment, the imaging direction of the form image data is the predetermined direction, and the imaging section of the form image data is the predetermined projection surface. The simplified PET image data generating unit 41 according to the present embodiment generates the simplified PET image data based on the detection position information detected in a direction corresponding to the imaging direction of the form image data among the detection position information of the gamma rays emitted from the subject 150. In other words, in the present embodiment, the simplified PET image data generating unit 41 generates the simplified PET image data based on the detection position information detected in a direction corresponding to a direction perpendicular to the imaging section of the form image data among the detection position information of the gamma rays emitted from the subject 150. The imaging direction of the form image data is a direction of the center of the X-ray beam in a cone beam shape or a fan beam shape output from the X-ray generating unit 11 in the scanogram imaging. In the description below, the imaging direction of the form image data may be referred to as a direction of the center of the X-ray irradiation direction.

The simplified PET image data generating unit 41 generates the simplified PET image data based on the detection position information of the gamma ray detected in the process of acquisition of the projection data in the PET imaging mode. In the present embodiment, the simplified PET image data generating unit 41 generates the simplified PET image data based on the detection position information of the gamma ray detected in the predetermined direction in the process of acquisition of the projection data in the PET imaging mode.

The simplified PET image data generating unit 41 generates the simplified PET image data by sequentially arranging a measuring point whose luminance decreases with the passage of time since the detection time in an address of a data sheet corresponding to the position of the generation source of the gamma ray. The simplified PET image data generating unit 41 according to the present embodiment generates the simplified PET image data by sequentially arranging the measuring point whose luminance decreases with the passage of time since the detection time in the address of the data sheet corresponding to the detection position of the gamma ray detected in the imaging direction of the form image data serving as the predetermined direction.

The simplified PET image data generating unit 41 includes a distribution data forming unit, an elapsed time measuring unit, and a look up table, which are not illustrated, for example. If the detection direction of the gamma ray measured by the detection direction measuring unit 222 of the detection data processing unit 22 is nearly equal to the direction of the center of the X-ray irradiation direction (e.g., the y-direction in FIG. 2) in the scanogram imaging mode, the simplified PET image data generating unit 41 sequentially arranges a measuring point having predetermined luminance in the address of the data sheet corresponding to the detection position of the gamma ray. Thus, the simplified PET image data generating unit 41 generates the simplified PET image data in which the distribution state of the gamma-ray generation source is projected onto the projection surface orthogonal to the direction of the center of the X-ray irradiation direction described above.

In other words, if the detector module 21 of the PET imaging unit 2 detects a gamma ray whose detection direction is a direction (y-direction) nearly equal to the direction of the center of the X-ray irradiation direction, the elapsed time measuring unit measures elapsed time from the detection time of the gamma ray detected by the detection time measuring unit 234 of the detection data processing unit 22 to the observation time. The look up table stores therein in advance corresponding data of the elapsed time and the luminance value of the measuring point in units of imaging conditions of the PET imaging mode including administered medical agent information. The elapsed time measuring unit extracts the luminance value corresponding to the measurement result of the elapsed time from the look up table.

The distribution data forming unit arranges a plurality of measuring points having luminance values that change with the passage of time in the address of the data sheet corresponding to the detection position of the gamma ray. Thus, the distribution data forming unit generates the simplified PET image data composed of the measuring points whose luminance reaches the maximum value at the detection time and decreases with the passage of time.

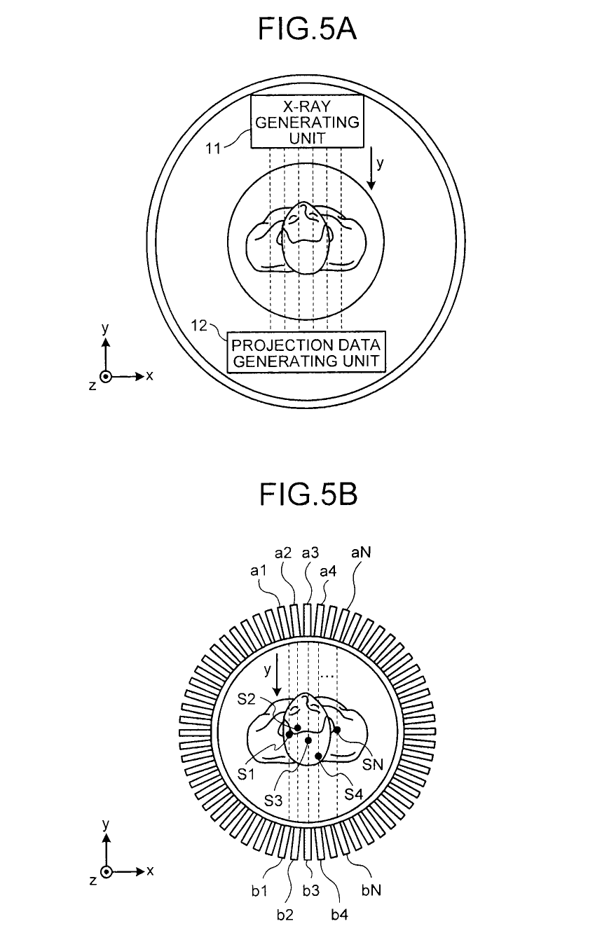

FIGS. 5A and 5B are schematics illustrating the detection directions and the detection positions of gamma rays in the simplified PET imaging mode according to the present embodiment. FIG. 5A illustrates the directions of the centers of the X-ray irradiation directions in the scanogram imaging mode. FIG. 5B illustrates detection positions a1 to aN and b1 to bN when the gamma rays emitted from inside of the body of the subject 150 are detected in directions nearly equal to the directions of the centers of the X-ray irradiation directions. To simplify the explanation, however, the direction of the center of the X-ray irradiation direction is the y-direction in FIG. 5A. Furthermore, in FIG. 5B, only the gamma-ray detection positions on one section perpendicular to the body axis direction are illustrated. Therefore, illustration of the gamma-ray detection positions on other sections perpendicular to the body axis direction is omitted. In the present embodiment, however, the gamma-ray detection positions on the other sections perpendicular to the body axis direction are also detected in the same manner.

In the example illustrated in FIG. 5A, the scanogram used as the form image data in the present embodiment is image data obtained by projecting a form of a tissue of the subject 150 onto the zx-plane in the y-direction. As illustrated in FIG. 5B, an LOR connecting the detection positional and the detection position b1 is a direction nearly equal to the y-direction. Therefore, the position obtained by projecting the position of a generation source S1 that is present at any position on the LOR onto the zx-plane in the y-direction is represented not only by the z-coordinate and the x-coordinate of the generation source S1, but also by the z-coordinate and the x-coordinate of the detection position a1 and the z-coordinate and the x-coordinate of the detection position b1.

The position obtained by projecting the position of a generation source S2 illustrated in FIG. 5B onto the zx-plane in the y-direction is represented by the z-coordinates and the x-coordinates of the detection position a2 and the detection position b2. The position obtained by projecting the position of a generation source S3 illustrated in FIG. 5B onto the zx-plane in the y-direction is represented by the z-coordinates and the x-coordinates of the detection position a3 and the detection position b3. The position obtained by projecting the position of a generation source S4 illustrated in FIG. 5B onto the zx-plane in the y-direction is represented by the z-coordinates and the x-coordinates of the detection position a4 and the detection position b4. The position obtained by projecting the position of a generation source SN illustrated in FIG. 5B onto the zx-plane in the y-direction is represented by the z-coordinates and the x-coordinates of the detection position aN and the detection position bN.

In other words, if the direction of the LOR is a direction nearly coincident with the direction (imaging direction) perpendicular to the imaging section, the position obtained by projecting the position of the generation source of the gamma ray onto the imaging section can be calculated from two pieces of detection position information used for determining the direction of the LOR without specifying the position of the generation source in the imaging direction. The imaging direction and the imaging section of the form image data are not limited to the y-direction and the zx-section, respectively, and can be changed arbitrarily by an operator. For example, by converting the information on the detection position measured by the detection position measuring unit 235 into positional information in a rectangular coordinate system specified by the imaging direction and the imaging section thus set, the simplified PET image data generating unit 41 can calculate the position obtained by projecting the position of the generation source of the gamma ray onto the imaging section. As described above, the simplified PET image data generating unit 41 generates the simplified PET image data in which the position of the generation source of the gamma ray on the imaging section is visualized without performing reconstruction processing.

Referring back to FIG. 1, the projection data storage unit 42 of the function image data generating unit 4 temporarily stores therein the projection data in the PET imaging mode generated by cumulative addition of the count value of a plurality of detection signals performed by the projection data generating unit 223 of the detection data processing unit 22 included in the PET imaging unit 2. The PET image data generating unit 43 uses the projection data in the PET imaging mode to generate PET image data. The PET image data generating unit 43 reconstructs the projection data in the PET imaging mode read from the projection data storage unit 42, thereby generating diagnostic PET image data.

The image data synthesis unit 5 includes an additive synthesis processing unit, which is not illustrated. The image data synthesis unit 5 synthesizes the scanogram having a broad area in the body axis direction stored in the scanogram storage unit 33 of the form image data generating unit 3 and the simplified PET image data generated in nearly real time by the simplified PET image data generating unit 41 of the function image data generating unit 4. Thus, the image data synthesis unit 5 generates evaluation image data used for evaluating an image quality deterioration factor included in the projection data in the PET imaging mode. In this case, the scanogram and the simplified PET image data are synthesized based on the imaging position information in the scanogram imaging mode (that is, positional information of the X-ray CT gantry) and the imaging position information in the simplified PET imaging mode (that is, positional information of the PET gantry).

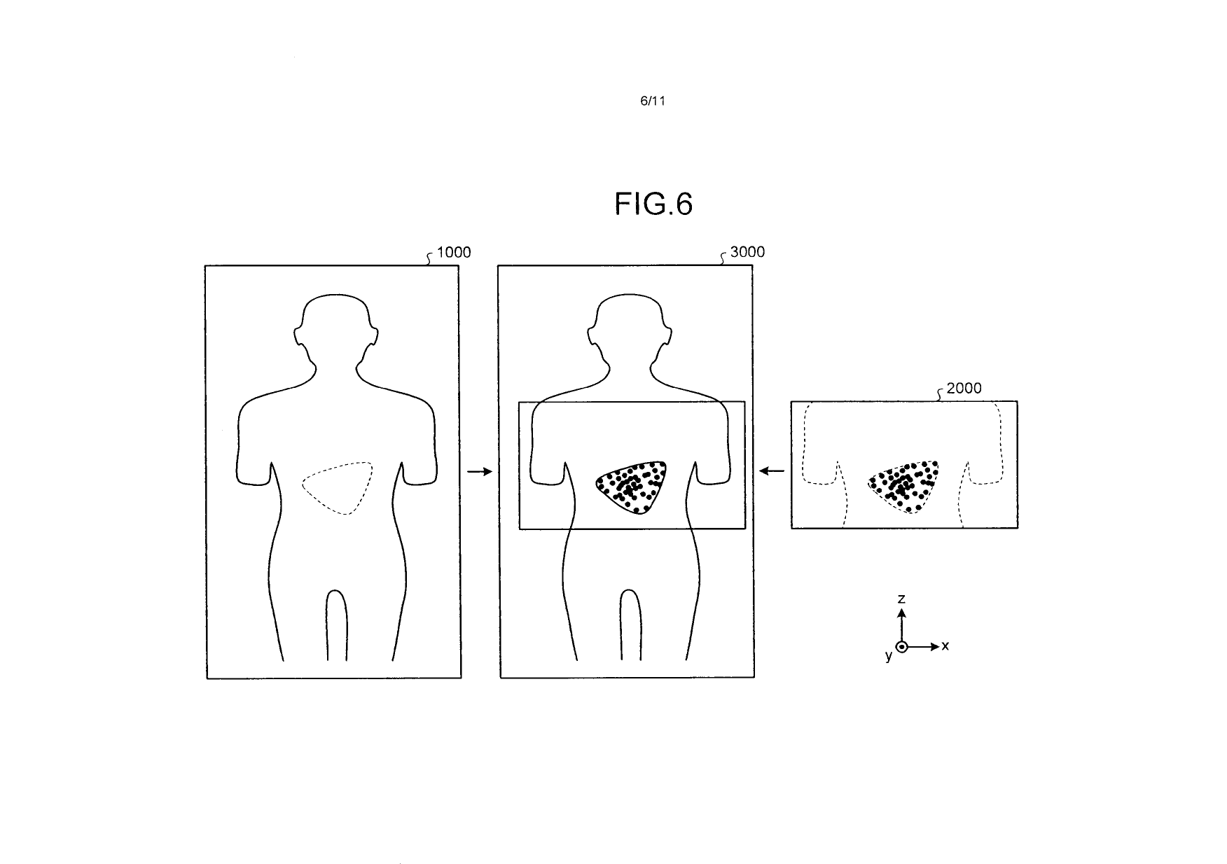

FIG. 6 is a schematic for explaining the evaluation image data generated by the image data synthesis unit according to the present embodiment. Image data 1000 illustrated in FIG. 6 is a scanogram of a broad area of the subject 150 generated by the scanogram generating unit 32 of the form image data generating unit 3 based on the projection data in the scanogram imaging mode acquired by X-ray irradiation centering on the y-direction indicated by an arrow in FIG. 5A. Image data 2000 illustrated in FIG. 6 is simplified PET image data generated by the simplified PET image data generating unit 41 of the function image data generating unit 4 based on the detection position of the gamma ray detected in a direction coincident with the direction of the center of the X-ray irradiation direction. Image data 3000 illustrated in FIG. 6 is evaluation image data generated by the image data synthesis unit 5 synthesizing the simplified PET image data and the scanogram.

In the evaluation image data displayed on the display 6, if each of the measuring points constituting the simplified PET image data is distributed to the inside of an organ to be examined indicated by a dashed line in the scanogram, for example, it is determined that influence of body movement during acquisition of the projection data in the PET imaging mode falls within an acceptable range.

The display 6 illustrated in FIG. 1 includes a display data generating unit, a conversion processing unit, and a monitor, which are not illustrated. The display data generating unit converts the evaluation image data generated by the image data synthesis unit 5 and the PET image data generated by the PET image data generating unit 43 of the function image data generating unit 4 into a predetermined display format, thereby generating display data. The conversion processing unit performs conversion, such as digital/analog (D/A) conversion and TV format conversion, on the display data generated by the display data generating unit, and displays the display data thus converted on the monitor. In the present embodiment, the display 6 displays, in real time, the simplified PET image data having a plurality of measuring points whose luminance decreases with the passage of time. Specifically, in the present embodiment, the display 6 displays the evaluation image data in real time.

The movement mechanism unit 8 includes a gantry rotating unit, a gantry moving unit, and a movement mechanism control unit, which are not illustrated. The gantry rotating unit rotates the rotating gantry 13 of the X-ray CT imaging unit 1 on which the X-ray tube 111 and the X-ray detector 121 are mounted in accordance with a gantry rotation control signal supplied from the movement mechanism control unit. Thus, the gantry rotating unit arranges the rotating gantry 13 to a position suitable for generating scanogram image data.

The gantry moving unit moves the X-ray CT gantry including the X-ray CT imaging unit 1 and the PET gantry including the PET imaging unit 2 along a guide rail provided to a floor surface in the body axis direction of the subject 150 in accordance with a gantry movement control signal supplied from the movement mechanism control unit.

The movement mechanism control unit supplies the gantry rotation control signal and the gantry movement control signal generated based on imaging conditions in the scanogram imaging mode and the PET imaging mode supplied from the input unit 9 via the system control unit 10 to the gantry rotating unit and the gantry moving unit, respectively.

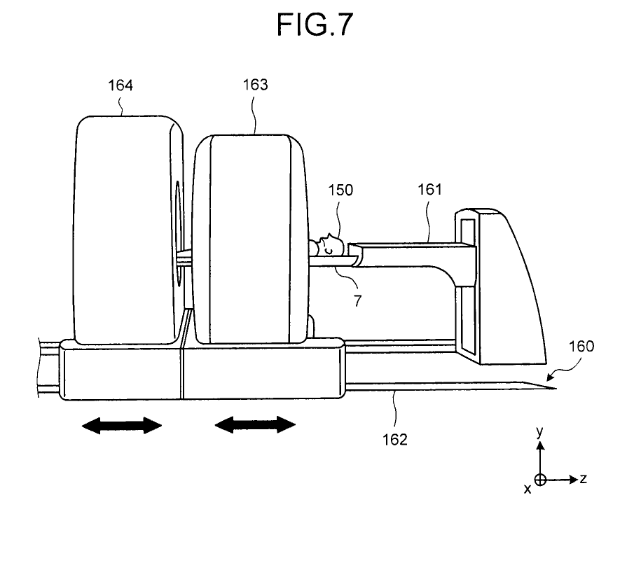

FIG. 7 is a schematic for explaining the X-ray CT gantry and the PET gantry moved by the movement mechanical unit according to the present embodiment. As illustrated in FIG. 7, a couch 161 including the couchtop 7 on which the subject 150 is placed is fixed to a floor surface 160 of a laboratory, and a guide rail 162 is arranged in the body axis direction (z-direction) of the couchtop 7. The movement mechanism unit 8 moves an X-ray CT gantry 163 including the X-ray CT imaging unit 1 and a PET gantry 164 including the PET imaging unit 2 along the guide rail 162 in the body axis direction such that the region to be examined (organ to be examined) of the subject 150 is arranged in an imaging field of the X-ray CT imaging unit 1 and an imaging field of the PET imaging unit 2. Thus, the imaging positions in the scanogram imaging mode and the PET imaging mode are specified.

The input unit 9 illustrated in FIG. 1 includes input devices, such as a keyboard, a selection switch, and a mouse, and a display panel, and forms an interactive interface in combination with the display 6. The input unit 9 receives various types of information by using the display panel and the input devices. Examples of the various types of information include the subject information, selection of the scanogram imaging mode, the simplified PET imaging mode, and the PET imaging mode, setting of the imaging conditions in these imaging modes, setting of the generating conditions and the display conditions for the scanogram, the simplified PET image data, and the PET image data, information on the radioisotope administered to the subject 150 (administered medical agent information), and various types of instruction signals, such as an X-ray CT imaging start instruction signal, a PET imaging start instruction signal, and a PET image data generation instruction signal. In the present embodiment, the input unit 9 receives an instruction signal for generating PET image data (PET image data generation instruction signal) based on evaluation results of the simplified PET image data. Specifically, in the present embodiment, the input unit 9 receives the PET image data generation instruction signal based on evaluation results of the evaluation image data. More specifically, the input unit 9 receives the PET image data generation instruction signal from the operator who observes the evaluation image data, and inputs the PET image data generation instruction signal thus received to the system control unit 10.

The system control unit 10 includes a central processing unit (CPU) and a storage circuit, which are not illustrated. The pieces of input information, selection information, and setting information supplied from the input unit 9 described above are stored in the storage circuit. Based on the information read from the storage circuit, the CPU collectively controls the units included in the medical image diagnostic apparatus 100, such that the units generate a scanogram, simplified PET image data, evaluation image data, and PET image data. For example, in accordance with the control performed by the system control unit 10 that receives the PET image data generation instruction signal from the input unit 6, the PET image data generating unit 43 reconstructs the projection data in the PET imaging mode, thereby generating PET image data.

Generation Process of PET Image Data

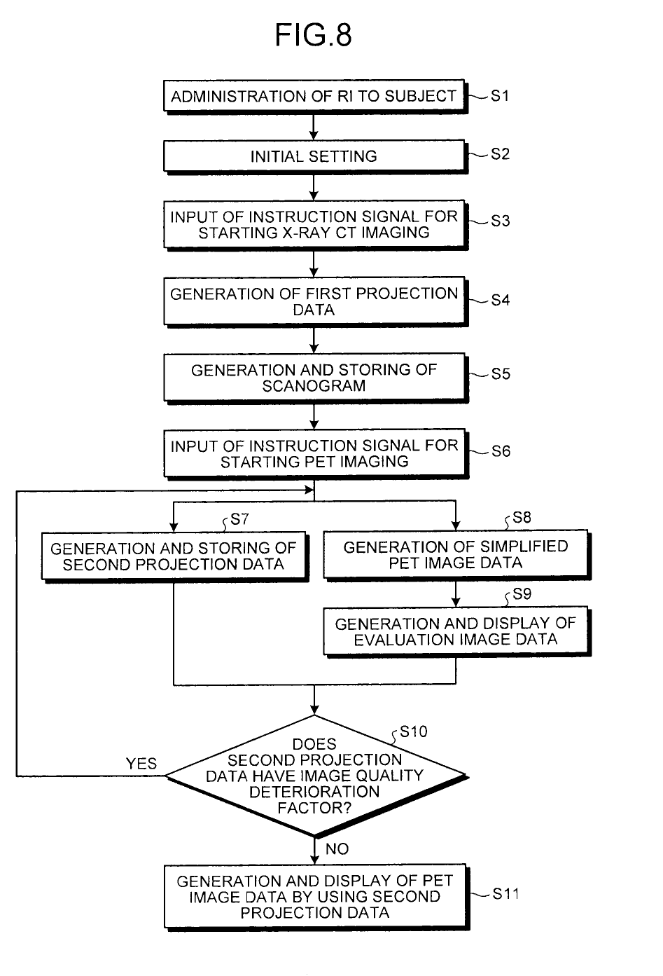

A generation process of PET image data according to the present embodiment will now be described with reference to a flowchart in FIG. 8. FIG. 8 is a flowchart of the generation process of PET image data according to the present embodiment.

Before performing X-ray CT imaging and PET imaging on the subject 150, the operator of the medical image diagnostic apparatus 100 administers a radioisotope (RI) labeled with a positron-emitting radionuclide such as ¹¹C, ¹³N, ¹⁵O, and ¹⁸F to the subject 150 (Step S1 in FIG. 8). The operator then performs initial setting (Step S2 in FIG. 8). In other words, the operator inputs the subject information and the administered medical agent information (e.g., the type of the RI, an applied dose V0, and administration time t0), sets the imaging conditions in the scanogram imaging mode and the PET imaging mode, and sets the generating conditions and the display conditions for a scanogram, simplified PET image data, evaluation image data, and PET image data in the input unit 9, for example. These pieces of input information and setting information are stored in the storage circuit of the system control unit 10.

Subsequently, the operator places the subject 150 on the couchtop 7. The operator then inputs a gantry movement instruction signal to the input unit 9, and moves the X-ray CT gantry 163 along the guide rail 162 in the body axis direction such that the subject 150 is arranged in the imaging field of the X-ray CT imaging unit 1. If the movement of the X-ray CT gantry 163 is completed, the operator inputs an instruction signal for starting X-ray CT imaging intended to generate a scanogram (X-ray CT imaging start instruction signal) to the input unit 9 (Step S3 in FIG. 8).

The system control unit 10 that receives the instruction signal performs generation of projection data (first projection data) in the scanogram imaging mode (Step S4 in FIG. 8). In other words, the system control unit 10 controls the units included in the X-ray CT imaging unit 1 based on the imaging conditions of the scanogram imaging mode read from the storage circuit thereof. Thus, the system control unit 10 performs X-ray imaging on the subject 150 successively moving in the body axis direction with the rotating gantry 13 on which the X-ray tube 111 and the X-ray detector 121 are mounted being fixed to a predetermined position. The projection data in the scanogram imaging mode acquired at this time is stored in the projection data storage unit 31 of the form image data generating unit 3 together with additional information, such as identification information of the imaging mode, and imaging position information (that is, positional information of the X-ray CT gantry 163).

The scanogram generating unit 32 of the form image data generating unit 3 performs generation and storing of the scanogram (Step S5 in FIG. 8). In other words, based on the imaging position information serving as the additional information, the scanogram generating unit 32 synthesizes the projection data in the scanogram imaging mode read from the projection data storage unit 31 based on the identification information of the imaging mode. The scanogram generating unit 32 then performs filtering for the purpose of noise reduction and edge enhancement as needed, for example, and generates a scanogram having a broad area in the body axis direction. The scanogram thus obtained is stored in the scanogram storage unit 33.

If generation and storing of the scanogram is completed by the process described above, the operator inputs a gantry movement instruction signal to the input unit 9 again, and moves the PET gantry 164 along the guide rail 162 in the body axis direction such that the region to be examined of the subject 150 is arranged in the imaging field of the PET imaging unit 2. If the movement of the PET gantry 164 is completed, the operator inputs an instruction signal for starting PET imaging intended to generate simplified PET image data and PET image data (PET imaging start instruction signal) to the input unit 9 (Step S6 in FIG. 8).

The system control unit 10 that receives the instruction signal controls the units included in the PET imaging unit 2 based on the imaging conditions of the PET imaging mode read from the storage circuit thereof, and starts PET imaging of the subject 150. Subsequently, the projection data generating unit 223 provided to the data processing unit 221 of the PET imaging unit 2 stores therein the count value of the detection signal supplied from the detection direction measuring unit 222 in a manner corresponding to the detection position and the detection direction of the gamma ray. At the same time, the projection data generating unit 223 cumulatively adds the count value of the detection signal detected at the same detection position and in the same detection direction in a predetermined time period sequentially. Thus, the projection data generating unit 223 generates projection data (second projection data) in the PET imaging mode, and stores the projection data thus obtained (second projection data) in the projection data storage unit 42 of the function image data generating unit 4 (Step S7 in FIG. 8).

By contrast, if the detection direction of the gamma ray measured by the detection direction measuring unit 222 is coincident with the direction of the center of the X-ray irradiation direction in the scanogram imaging mode, the simplified PET image data generating unit 41 of the function image data generating unit 4 sequentially arranges a measuring point whose luminance decreases with the passage of time in an address of a data sheet corresponding to the detection position of the gamma ray. Thus, the simplified PET image data generating unit 41 generates simplified PET image data in which the distribution state of the gamma-ray generation source is projected onto the projection surface orthogonal to the X-ray irradiation direction (Step S8 in FIG. 8).

Subsequently, the image data synthesis unit 5 synthesizes the scanogram having a broad area in the body axis direction stored in the scanogram storage unit 33 of the form image data generating unit 3 and the simplified PET image data generated in nearly real time by the simplified PET image data generating unit 41 of the function image data generating unit 4. Thus, the image data synthesis unit 5 generates evaluation image data, and displays the evaluation image data on the monitor of the display 6 (Step S9 in FIG. 8).

The operator of the medical image diagnostic apparatus 100 observes the evaluation image data displayed on the display 6. Based on the evaluation image data, the operator determines whether an image quality deterioration factor is present in the projection data (second projection data) in the PET imaging mode that has already been acquired (Step S10 in FIG. 8). If it is determined that no image quality deterioration factor is present (NO at Step S10 in FIG. 8), the operator inputs a PET image data generation instruction signal to the input unit 9. The PET image data generating unit 43 of the function image data generating unit 4 then receives the instruction signal via the system control unit 10. The PET image data generating unit 43 then reconstructs the projection data in the PET imaging mode read from the projection data storage unit 42, thereby generating diagnostic PET image data. The PET image data thus obtained is displayed on the monitor of the display 6 (Step S11 in FIG. 8). At Step S11, the display 6 may display the PET image data, or may display the evaluation image data and the PET image data.

By contrast, if it is determined that an unacceptable image quality deterioration factor is present in the projection data in the PET imaging mode by observing the evaluation image data generated at Step S9 (YES at Step S10 in FIG. 8), the operator inputs an instruction signal for reacquiring the projection data to the input unit 9. The system control unit 10 that receives the instruction signal controls the units included in the PET imaging unit 2 to repeat the processing at Step S7 to Step S9. Thus, the system control unit 10 causes the units to generate new projection data and to generate and display new evaluation image data.

In the process described above, the RI is administered to the subject 150 before the start of the X-ray CT imaging. Alternatively, the RI may be administered when the X-ray CT imaging is completed. Furthermore, to perform whole-body PET imaging of the subject 150 by the step and shoot method in the present embodiment, the PET gantry 164 is moved such that a region to be examined partially overlapping with the region to be examined in the PET image data generated at Step S11 is arranged in the imaging field of the PET imaging unit 2 after Step S11. Subsequently, the processing after Step S7 is performed.

Modifications

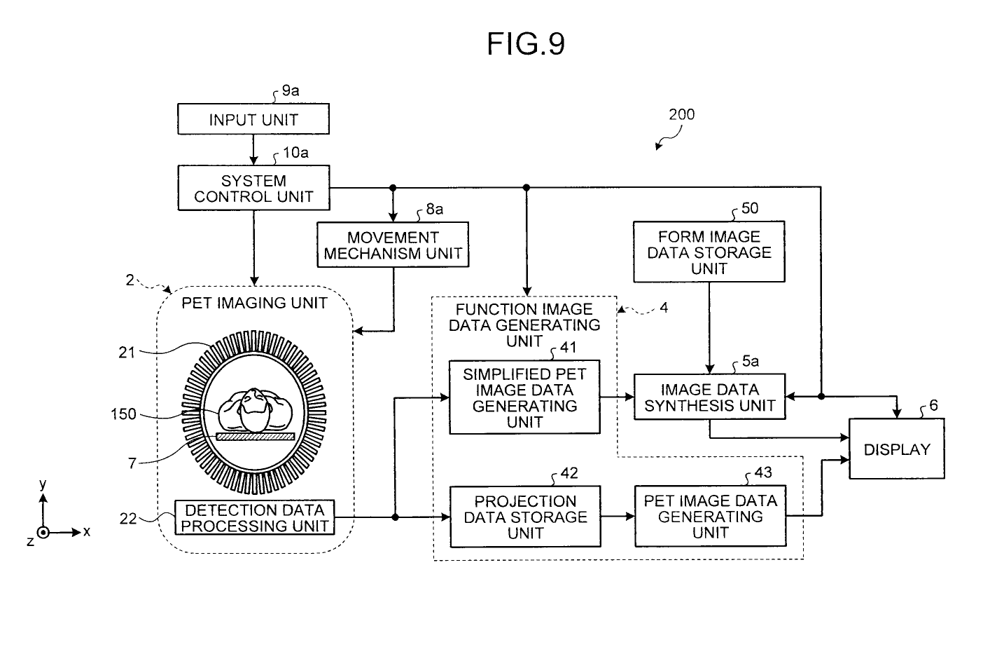

A modification of the present embodiment will now be described with reference to FIG. 9. FIG. 9 is a block diagram of an entire configuration of a medical image diagnostic apparatus according to a modification of the present embodiment.

In the embodiment, the explanation has been made of the medical image diagnostic apparatus capable of generating a scanogram serving as form image data and PET image data serving as function image data. In the present modification, only a medical image diagnostic apparatus that generates the PET image data is explained.

In other words, the medical image diagnostic apparatus according to the present modification stores form image data of the subject 150 generated by another medical image diagnostic apparatus in a form image data storage unit. Subsequently, the medical image diagnostic apparatus according to the present modification generates projection data in the PET imaging mode based on the detection direction and the detection position of a gamma ray emitted from inside of the body of the subject 150 to whom a radioisotope is administered. At the same time, the medical image diagnostic apparatus generates simplified PET image data based on the detection position of a gamma ray detected in a direction nearly equal to the imaging direction of the form image data or to a direction perpendicular to the imaging section extracted from the detection result of the gamma ray. The medical image diagnostic apparatus according to the present modification then superimposes the simplified PET image data thus obtained on the form image data read from the form image data storage unit, thereby generating evaluation image data. If it is determined that no unacceptable image quality deterioration factor is included in the projection data in the PET imaging mode by observing the evaluation image data in which the detection position of the gamma ray is displayed real time on a display, the medical image diagnostic apparatus according to the present modification reconstructs the projection data, thereby generating diagnostic PET image data.

In the block diagram of FIG. 9 illustrating the entire configuration of the medical image diagnostic apparatus according to the present modification, units having the same configurations and functions as those of the units in the medical image diagnostic apparatus 100 illustrated in FIG. 1 are represented by similar reference numerals. Furthermore, detailed explanations thereof will be omitted.

In other words, a medical image diagnostic apparatus 200 according to the present modification illustrated in FIG. 9 includes a PET imaging unit 2, a form image data storage unit 50, a function image data generating unit 4, an image data synthesis unit 5a, and a display 6. The PET imaging unit 2 causes detector modules 21 arranged around the subject 150 to detect a pair of gamma rays emitted from inside of the body of the subject 150 to whom the radioisotope is administered, thereby generating projection data in the PET imaging mode based on the detection directions and the detection positions of the gamma rays. The form image data storage unit 50 stores therein in advance form image data acquired by an external medical image diagnostic apparatus, for example. The function image data generating unit 4 generates simplified PET image data based on the detection position of the gamma ray detected in a direction nearly equal to the imaging direction of the form image data or to a direction perpendicular to the imaging section. Furthermore, the function image data generating unit 4 generates PET image data serving as function image data based on the projection data in the PET imaging mode generated by the PET imaging unit 2. The image data synthesis unit 5a superimposes the simplified PET image data on the form image data, thereby generating evaluation image data used for evaluating an image quality deterioration factor in the projection data in the PET imaging mode. The display 6 displays the evaluation image data generated by the image data synthesis unit 5a and the PET image data generated by the function image data generating unit 4.

The medical image diagnostic apparatus 200 further includes a couchtop 7 on which the subject 150 is placed and a movement mechanical unit 8a. The movement mechanical unit 8a moves a PET gantry, which is not illustrated, including the PET imaging unit 2 in the body axis direction (z-direction in FIG. 9), thereby arranging a region to be examined of the subject 150 in the imaging field of the PET imaging unit 2. The medical image diagnostic apparatus 200 further includes an input unit 9a and a system control unit 10a. The input unit 9a receives subject information, selection of the simplified PET imaging mode and the PET imaging mode, setting of imaging conditions in these imaging modes, setting of generating conditions and display conditions for the simplified PET image data, the evaluation image data, and the PET image data, and various types of command signals, for example. The system control unit 10a collectively controls the units included in the medical image diagnostic apparatus 200.

The form image data storage unit 50 stores therein in advance a scanogram generated by an X-ray CT apparatus and a magnetic resonance imaging (MRI) apparatus, MPR image data at a coronal section of the subject 150, and transparent image data generated by an X-ray diagnostic apparatus, for example, together with imaging position information serving as additional information.

The image data synthesis unit 5a includes an additive synthesis processing unit, which is not illustrated. The image data synthesis unit 5a synthesizes the form image data having a broad area in the body axis direction stored in the form image data storage unit 50 and the simplified PET image data generated in nearly real time by a simplified PET image data generating unit 41 of the function image data generating unit 4. Thus, the image data synthesis unit 5a generates evaluation image data used for evaluating an image quality deterioration factor included in the projection data in the PET imaging mode. In this case, the form image data and the simplified PET image data are synthesized based on the imaging position information added to each image data.

The movement mechanism unit 8a includes a gantry moving unit and a movement mechanism control unit, which are not illustrated. The gantry moving unit moves the PET gantry including the PET imaging unit 2 along a guide rail provided to a floor surface in the body axis direction of the subject 150 in accordance with a gantry movement control signal supplied from the movement mechanism control unit. The movement mechanism control unit supplies a gantry movement control signal generated based on imaging conditions of the PET imaging mode supplied from the input unit 9a via the system control unit 10a to the gantry moving unit.

The input unit 9a includes input devices, such as a keyboard, a selection switch, and a mouse, and a display panel, and forms an interactive interface in combination with the display 6. The input unit 9a receives various types of information by using the display panel and the input devices. Examples of the various types of information include the subject information, selection of the simplified PET imaging mode and the PET imaging mode, setting of the imaging conditions in these imaging modes, setting of the generating conditions and the display conditions for the simplified PET image data and the PET image data, information on the radioisotope administered to the subject 150 (administered medical agent information), and various types of instruction signals, such as a PET imaging start instruction signal and a PET image data generation instruction signal.

The system control unit 10a includes a CPU and a storage circuit, which are not illustrated. The pieces of input information, selection information, and setting information supplied from the input unit 9a described above are stored in the storage circuit. Based on these pieces of information read from the storage circuit, the CPU collectively controls the units included in the medical image diagnostic apparatus 200, such that the units generate simplified PET image data, evaluation image data, and PET image data.

A generation process of PET image data in the present modification is the same as the process from Step S6 to Step S10 illustrated in FIG. 8. Therefore, an explanation thereof will be omitted.

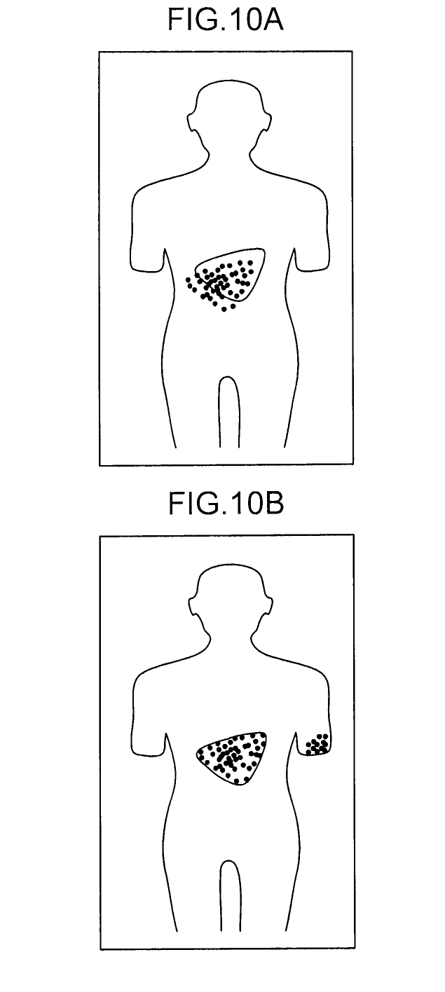

A specific example of an image quality deterioration factor determined by the evaluation image data of the present embodiment and the modification will now be described with reference to FIG. 10A and FIG. 10B. FIG. 10A and FIG. 10B are schematics for explaining a specific example of an image quality deterioration factor determined by the evaluation image data of the present embodiment and the modification thereof.

FIG. 10A illustrates evaluation image data obtained when a prominent image quality deterioration factor due to body movement of the subject 150 occurs in projection data in the PET imaging mode. In this case, a part of a plurality of measuring points in the simplified PET image data is displayed outside of an organ to be examined in form image data such as a scanogram. Therefore, the operator can determine whether to reacquire the projection data based on the positions and the number of the measuring points displayed near the organ to be examined in the simplified PET image data.

By contrast, FIG. 10B illustrates evaluation image data obtained when an image quality deterioration factor due to medical agent leakage at a medical agent injection site during administration of the radioisotope to the subject 150 occurs in projection data in the PET imaging mode. In this case, many measuring points indicating the generation sources of the gamma rays are displayed near the medical agent injection site on an upper limb of the subject in the form image data. If many gamma rays due to medical agent leakage and the like are detected in a region other than the organ to be examined, these gamma rays function as the image quality deterioration factor, thereby making it difficult to obtain excellent PET image data. In such a case, the operator can determine whether to reacquire the projection data based on the positions, the number, and the frequency of the measuring points displayed in the region other than the organ to be examined in the evaluation image data, for example.

According to the present embodiment and the modification, when projection data is generated based on detection information of a gamma ray emitted from a subject to whom a radioisotope is administered, and PET image data is generated by reconstructing the projection data, it is possible to determine whether or how much an image quality deterioration factor affects the projection data in a short time by using simplified PET image data generated based on a gamma ray detected in a predetermined direction.

In particular, by synthesizing form image data such as a scanogram acquired from the subject and the simplified PET image data, and displaying the data thus synthesized, it is possible to facilitate recognition of influence due to body movement of the subject occurring when the projection data is acquired, influence of medical agent leakage occurring when the radioisotope is administered, and other influence accurately.

Furthermore, in the simplified PET image data, a measuring point whose luminance reaches the maximum value at the detection time and decreases with the passage of time is displayed in real time. Therefore, it is possible to recognize temporal change in the image quality deterioration factor due to the body movement, the medical agent leakage, and the like in real time.

In other words, according to the present embodiment and the modification, by observing in real time the simplified PET image data generated based on the detection position information of the gamma ray detected in the predetermined direction when the PET image data is generated based on the projection data in the PET imaging mode, it is possible to determine whether or how much various types of image quality deterioration factors affect the projection data. According to the present embodiment and the modification, if an unacceptable image quality deterioration factor is present in the projection data, the projection data of the subject can be reacquired prior to generation of the PET image data.

Accordingly, excellent PET image data can be generated constantly, thereby improving the diagnostic accuracy. Furthermore, if an image quality deterioration factor occurs, the projection data is reacquired immediately. As a result, the subject need not be readministered the radioisotope or come back to the hospital, for example. Therefore, it is possible not only to improve the examination efficiency significantly, but also to reduce the burden on the subject.

While certain embodiments and modifications have been described, these embodiments and modifications have been presented by way of example only, and are not intended to limit the scope of the description. Indeed, the description described herein may be embodied in a variety of other forms. In the embodiment, for example, the explanation has been made of the medical image diagnostic apparatus capable of generating a scanogram serving as form image data based on projection data in the scanogram imaging mode and PET image data serving as function image data based on projection data in the PET imaging mode. Alternatively, in the present embodiment, three-dimensional image data generated based on projection data in the X-ray CT imaging mode obtained by rotating the X-ray tube 111 and the X-ray detector 121 around the subject 150 at high speed or two-dimensional image data at a predetermined section (e.g., a coronal section) may be used as the form image data. The two-dimensional data in this case may be MPR image data or maximum intensity projection (MIP) image data based on three-dimensional data (volume data) obtained by reconstructing the projection data in the X-ray CT imaging mode.

In the embodiment, by moving the X-ray CT gantry 163 and the PET gantry 164 along the guide rail 162 in the body axis direction, the region to be examined of the subject 150 is arranged in the imaging field of the X-ray CT imaging unit 1 and the imaging field of the PET imaging unit 2. Alternatively, in the embodiment, by moving the couchtop 7 on which the subject 150 is placed in the body axis direction, the region to be examined may be arranged in the imaging fields.

In the embodiment and the modification, by using the evaluation image data generated by synthesizing the form image data such as a scanogram and the simplified image data, an image quality deterioration factor in the projection data in the PET imaging mode is evaluated. Alternatively, the image quality deterioration factor may be evaluated by using the simplified PET image data. In simplified PET image data obtained when medical agent leakage occurs, for example, many measuring points are displayed at a region distant from a region estimated to be an organ to be examined as illustrated in FIG. 10B. Therefore, to determine whether medical agent leakage occurs as an image quality deterioration factor, the simplified PET image data may be displayed as the evaluation image data in the embodiment and the modification. In such a case, the PET image data is generated based on the evaluation result of the simplified PET image data.

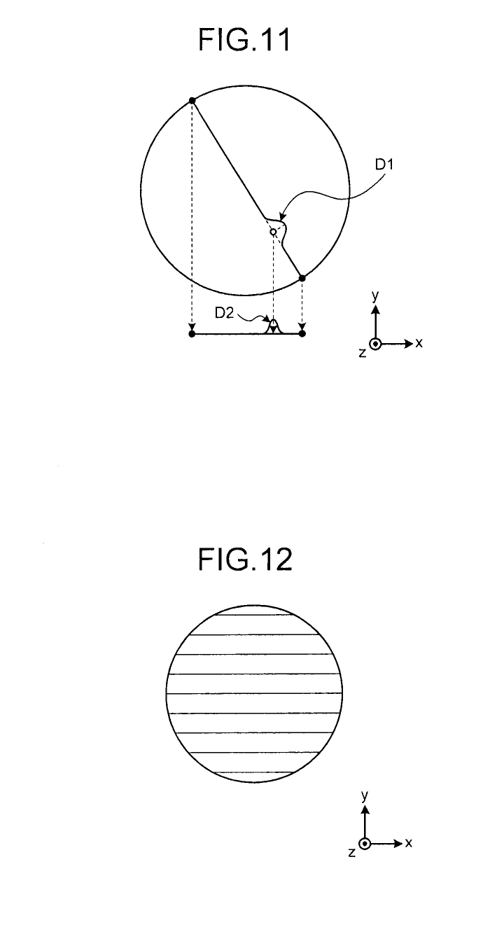

In the embodiment and the modification, the simplified PET image data is generated by using the detection position information in which the direction of an LOR is a predetermined direction as information obtained by projecting the position of the generation source of the gamma ray onto a predetermined projection surface in a predetermined direction. Alternatively, the simplified PET image data may be generated by a modification described below. FIG. 11 and FIG. 12 are schematics for explaining a modification of simplified PET image data generation processing.

As described above, a PET apparatus determines two detection positions the difference between the detection times of which is a predetermined duration to be positions at which a pair of gamma rays is detected nearly simultaneously, thereby generating projection data for reconstructing PET image data. In recent years, time of flight (TOF)-PET apparatuses that estimate the position of the generation source of the gamma ray on an LOR by using the difference between the detection times have been in practical use. In other words, as illustrated in FIG. 11, such a TOF-PET apparatus can estimate probability distribution D1 in which the gamma-ray generation source is present at each position on the LOR by using the detection time difference. Therefore, if the medical image diagnostic apparatus 100 or the medical image diagnostic apparatus 200 is a TOF-PET apparatus, the simplified PET image data generating unit 41 generates the simplified PET image data by using the position of the generation source of the gamma ray estimated based on the detection position information and the detection time information of the gamma ray.

As illustrated in FIG. 11, for example, the simplified PET image data generating unit 41 calculates probability distribution D2 obtained by projecting the probability distribution D1 on the LOR onto the zx-plane serving as the imaging section in the y-direction. The simplified PET image data generating unit 41 determines the position of the peak of the probability distribution D2 to be the measuring point, thereby generating the simplified PET image data, for example.

In the modification using the TOF function, an LOR extending in any direction can be used for the simplified PET image data. In the modification using the TOF function, for example, the simplified PET image data can be generated by using all LORs sequentially specified in a predetermined time period. As a result, in the modification using the TOF function, it is possible to evaluate the presence or the degree of the image quality deterioration factor accurately. In the modification, however, the position of the generation source of the gamma ray is estimated by using the detection time difference. As a result, the real-time property in generation and display of the simplified PET image data slightly deteriorates compared with the method using the LOR in the predetermined direction.

Therefore, when the TOF function is used, the simplified PET image data generating unit 41 may limit the LOR used for generating the simplified PET image data. If the predetermined direction is the y-direction, for example, the simplified PET image data generating unit 41 may limit the LOR used for generating the simplified PET image data to the LOR included in the xy-plane. The simplified PET image data generating unit 41, for example, may limit the LOR used for generating the simplified PET image data to the LOR that is included in the xy-plane and whose direction is in a predetermined directional range on the xy-plane. As illustrated in FIG. 12, the simplified PET image data generating unit 41, for example, may limit the LOR used for generating the simplified PET image data to the LOR whose direction is nearly equal to the x-direction. Furthermore, the simplified PET image data generating unit 41 may combine the method using the TOF function and the method using the LOR whose direction is nearly equal to the predetermined direction, thereby generating the simplified PET image data.

A part of the medical image diagnostic apparatus 100 according to the present embodiment and the medical image diagnostic apparatus 200 according to the modification thereof can also be realized by using a computer as hardware. In terms of the system control unit 10 (10a) included in the medical image diagnostic apparatus 100 (200), for example, various functions can be realized by causing a processor such as a CPU mounted on the computer to execute a predetermined control program. In this case, the system control unit 10 (10a) and other components may be realized by installing the control program on the computer in advance. Alternatively, the system control unit 10 (10a) and other components may be realized by storing the control program in a computer-readable storage medium, or installing the control program distributed via a network on the computer.

As described above, according to the embodiment and the modification, it is possible to determine whether or how much an image quality deterioration factor affects projection data acquired by PET imaging in a short time.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnostic apparatus comprising:
a simplified positron emission computed tomography (PET) image data generating unit configured to generate simplified PET image data based on information obtained by projecting a position of a generation source of a gamma ray emitted from a subject to whom a radioisotope is administered onto a predetermined projection surface in a predetermined direction;
a PET image data generating unit configured to generate, based on an evaluation result of the simplified PET image data, PET image data by using projection data in a PET imaging mode generated based on a detection result of the gamma ray emitted from the subject; and
a display configured to display the simplified PET image data and the PET image data.

2. The medical image diagnostic apparatus according to claim 1, further comprising:
an image data synthesis unit configured to superimpose the simplified PET image data on form image data acquired from the subject and to generate evaluation image data, wherein
the PET image data generating unit is configured to generate the PET image data based on an evaluation result of the evaluation image data, and
the display is configured to display the evaluation image data and the PET image data.

3. The medical image diagnostic apparatus according to claim 2, wherein the simplified PET image data generating unit is configured to generate the simplified PET image data based on detection position information detected in a direction corresponding to an imaging direction of the form image data or corresponding to a direction perpendicular to an imaging section among detection position information of the gamma ray emitted from the subject.

4. The medical image diagnostic apparatus according to claim 3, further comprising:
an X-ray computed tomography (CT) imaging unit configured to acquire projection data in an X-ray CT imaging mode from the subject; and
a form image data generating unit configured to generate at least one of a scanogram in the imaging direction and multi-planer reconstruction (MPR) image data at the imaging section as the form image data based on the projection data in the X-ray CT imaging mode acquired from the subject by the X-ray CT imaging unit.

5. The medical image diagnostic apparatus according to claim 3, wherein the image data synthesis unit is configured to superimpose the simplified PET image data on at least one of a scanogram in the imaging direction and multi-planer reconstruction (MPR) image data at the imaging section acquired from the subject by an external X-ray computed tomography (CT) apparatus or magnetic resonance imaging (MRI) apparatus and to generate the evaluation image data.

6. The medical image diagnostic apparatus according to claim 1, wherein the simplified PET image data generating unit is configured to generate the simplified PET image data based on detection position information of the gamma ray detected in a process of acquisition of the projection data in the PET imaging mode.

7. The medical image diagnostic apparatus according to claim 1, wherein the simplified PET image data generating unit is configured to generate the simplified PET image data by sequentially arranging a measuring point whose luminance decreases with passage of time since detection time in an address of a data sheet corresponding to the position of the generation source of the gamma ray.

8. The medical image diagnostic apparatus according to claim 7, wherein the display is configured to display, in real time, the simplified PET image data having a plurality of measuring points whose luminance decreases with the passage of time.

9. The medical image diagnostic apparatus according to claim 1, further comprising:
an instruction signal input unit configured to receive an instruction signal for generating the PET image data based on the evaluation result of the simplified PET image data, wherein
the PET image data generating unit is configured to reconstruct the projection data in the PET imaging mode in accordance with the instruction signal and to generate the PET image data.

10. The medical image diagnostic apparatus according to claim 1, wherein the simplified PET image data generating unit is configured to generate the simplified PET image data by using the position of the generation source of the gamma ray estimated based on detection position information and detection time information of the gamma ray.

11. A control method comprising:
generating, by a simplified positron emission computed tomography (PET) image data generating unit, simplified PET image data based on information obtained by projecting a position of a generation source of a gamma ray emitted from a subject to whom a radioisotope is administered onto a predetermined projection surface in a predetermined direction;
generating, by a PET image data generating unit, based on an evaluation result of the simplified PET image data, PET image data by using projection data in a PET imaging mode generated based on a detection result of the gamma ray emitted from the subject; and
displaying, by a display, the simplified PET image data and the PET image data.

12. The control method according to claim 11, further comprising:
superimposing, by an image data synthesis unit, the simplified PET image data on form image data acquired from the subject and generating evaluation image data, wherein
the PET image data generating unit is configured to generate the PET image data based on an evaluation result of the evaluation image data, and
the display is configured to display the evaluation image data and the PET image data.

13. The control method according to claim 12, wherein the simplified PET image data generating unit is configured to generate the simplified PET image data based on detection position information detected in a direction corresponding to an imaging direction of the form image data or corresponding to a direction perpendicular to an imaging section among detection position information of the gamma ray emitted from the subject.

14. The control method according to claim 13, further comprising:
acquiring, by an X-ray computed tomography (CT) imaging unit, projection data in an X-ray CT imaging mode from the subject; and
generating, by a form image data generating unit, at least one of a scanogram in the imaging direction and multi-planer reconstruction (MPR) image data at the imaging section as the form image data based on the projection data in the X-ray CT imaging mode acquired from the subject by the X-ray CT imaging unit.

15. The control method according to claim 13, wherein the image data synthesis unit is configured to superimpose the simplified PET image data on at least one of a scanogram in the imaging direction and multi-planer reconstruction (MPR) image data at the imaging section acquired from the subject by an external X-ray computed tomography (CT) apparatus or magnetic resonance imaging (MRI) apparatus and to generate the evaluation image data.

16. The control method according to claim 11, wherein the simplified PET image data generating unit is configured to generate the simplified PET image data based on detection position information of the gamma ray detected in a process of acquisition of the projection data in the PET imaging mode.

17. The control method according to claim 11, wherein the simplified PET image data generating unit is configured to generate the simplified PET image data by sequentially arranging a measuring point whose luminance decreases with passage of time since detection time in an address of a data sheet corresponding to the position of the generation source of the gamma ray.

18. The control method according to claim 17, wherein the display is configured to display, in real time, the simplified PET image data having a plurality of measuring points whose luminance decreases with the passage of time.

19. The control method according to claim 11, further comprising:
receiving, by an instruction signal input unit, an instruction signal for generating the PET image data based on the evaluation result of the simplified PET image data, wherein
the PET image data generating unit is configured to reconstruct the projection data in the PET imaging mode in accordance with the instruction signal and to generate the PET image.

20. The control method according to claim 11, wherein the simplified PET image data generating unit is configured to generate the simplified PET image data by using the position of the generation source of the gamma ray specified based on detection position information and detection time information of the gamma ray.
